(19) United States
(12) Patent Application Publication
(10) Pub. No.: US 2025/0201003 A1
(43) Pub. Date: Jun. 19, 2025

(54) METHODS AND SYSTEMS FOR INTEGRATING HIGH-THROUGHPUT CELLULAR PROFILES USING CELL CYCLE STATES

(71) Applicant: H. Lee Moffitt Cancer Center and Research Institute, Inc., Tampa, FL (US)

(72) Inventor: Noemi Andor, Wesley Chapel, FL (US)

(21) Appl. No.: 18/772,456

(22) Filed: Jul. 15, 2024

Related U.S. Application Data
(60) Provisional application No. 63/513,430, filed on Jul. 13, 2023.

(51) Int. Cl. G06V 20/69; G06T 7/00; G06V 10/62; G06V 10/762; G06V 10/77; G16B 30/00

(52) U.S. Cl. CPC G06V 20/698; G06T 7/0016; G06V 10/62; G06V 10/763; G06V 10/7715; G06V 20/695; G16B 30/00; G06T 2207/20081; G06T 2207/30024; G06T 2207/30242

(57) ABSTRACT

A system may receive sequencing data for a cell sample, the cell sample comprising a plurality of cells. A system may receive an image of the cell sample. A system may analyze the image to determine a plurality of respective cell cycle states for the plurality of cells in the cell sample. A system may integrate the sequencing data with the image using the plurality of respective cell cycle states.

### CROSS-REFERENCE TO RELATED APPLICATIONS

This application claims the benefit of U.S. provisional patent application No. 63/513,430, filed on Jul. 13, 2023, and titled “INTEGRATING IMAGING AND SEQUENCING TO COMPUTE THE SUBCELLULAR ORGANIZATION OF A CELL'S TRANSCRIPTOME,” the disclosure of which is expressly incorporated herein by reference in its entirety.

### STATEMENT REGARDING FEDERALLY FUNDED RESEARCH

This invention was made with government support under Grant no. CA259873 awarded by the National Institutes of Health. The government has certain rights in the invention.

### BACKGROUND

Understanding the cell cycle is fundamental to numerous areas of biological research and medical practice. The cell cycle consists of distinct phases: G1 Phase, S Phase, G2 Phase, M Phase, and G0 Phase, each characterized by specific cellular activities and morphological changes. Accurate identification of these phases is crucial for studying cell proliferation, differentiation, and response to treatments. Several methods for cell cycle inference from sequencing data exist and are widely adopted. In contrast, methods for classification of cell cycle state from imaging data are scarce.

### SUMMARY

In some aspects, the techniques described herein relate to a computer-implemented method including: receiving sequencing data for a cell sample, the cell sample including a plurality of cells; receiving an image of the cell sample; analyzing the image to determine a plurality of respective cell cycle states for the plurality of cells in the cell sample; and integrating the sequencing data with the image using the plurality of respective cell cycle states.

In some aspects, the step of integrating the sequencing data with the image includes: mapping each of the plurality of cells in the image to a set of the plurality of cells in the sequencing data using the plurality of respective cell cycle states; and mapping each of the plurality of cells in the sequencing data to a set of the plurality of cells in the image using the plurality of respective cell cycle states.

In some aspects, the image is a brightfield image.

In some aspects, the step of analyzing the image to determine the plurality of respective cell cycle states for the plurality of cells in the cell sample includes using a trained machine learning model.

In some aspects, the step of using the trained machine learning model includes: inputting the brightfield image into the trained machine learning model; and a spatial distribution of organelles of the plurality of cells in a simulated image of the cell sample from the trained machine learning model.

In some aspects, the computer-implemented method further includes segmenting one or more organelles of the plurality of cells in the simulated image of the cell sample.

In some aspects, the computer-implemented method further includes quantifying a plurality of cell features of the plurality of cells in the simulated image of the cell sample.

In some aspects, the plurality of cell features include area of cell, area of nucleus, number of cytoplasm density-based clustering algorithm (DBSCAN) clusters, number of mitochondria DBSCAN clusters, maximum area of available cross sections of the nucleus, ratio of nuclear volume to nuclear area, total pixel count of cell, total pixel count of mitochondria, total pixel count of nucleus, volume of cell, and volume of nucleus.

In some aspects, the computer-implemented method further includes correlating the plurality of cell features with a cell cycle state.

In some aspects, the step of correlating the plurality of cell features with the cell cycle state includes inferring a cell cycle pseudotime for a cell using one or more of the plurality of cell features, wherein the plurality of cell features are correlated with the cell cycle state using the cell cycle pseudotime.

In some aspects, the computer-implemented method further includes: providing a training dataset including brightfield images and corresponding fluorescent images; and training a machine learning model to training a machine learning model to predict spatial distributions of organelles of cells in simulated images using the training dataset.

In some aspects, the image is a fluorescently-labeled image.

In some aspects, the plurality of respective cell cycle states include one or more of G1 Phase, S Phase, G2 Phase, M Phase, and G0 Phase.

In some aspects, the techniques described herein relate to a method including: integrating sequencing data for a cell sample with an image as described herein; and providing a diagnosis, prognosis, or treatment recommendation for a subject based on the integrated sequencing data and image of the cell sample.

In some aspects, the techniques described herein relate to a method including: integrating sequencing data for a cell sample with an image as described herein; and administering a treatment to a subject based on the integrated sequencing data and image of the cell sample.

In some aspects, the techniques described herein relate to a computer system including: one or more processors and one or more computer-readable memories operably coupled to the one or more processors, the one or more computer-readable memories having instructions stored thereon that, when executed by the one or more processors, cause the computer system to perform a method including: receiving sequencing data for a cell sample, the cell sample including a plurality of cells; receiving an image of the cell sample; analyzing the image to determine a plurality of respective cell cycle states for the plurality of cells in the cell sample; and integrating the sequencing data with the image using the plurality of respective cell cycle states.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating example operations for integrating imaging and sequencing data using cell cycle states according to implementations described herein.

FIG. 2 is an example computing device.

FIGS. 3A-3F illustrate using FUCCI to characterize discrete and continuous cell cycle progression in NCI-N87 according to Example 1. FIG. 3A: Green and red Fucci channels overlayed. FIG. 3B, FIG. 3C: Fucci based discrete cell cycle classification. FIG. 3D: Fucci based pseudotime inference quantifies continuous cell cycle progression. FIG. 3E: Fucci based pseudotime trajectory in PCA space. Every dot represents a cell. FIG. 3F: Pseudotime is differentially distributed across the four discrete cell cycle classes. Data and code to produce this figure can be found on the project GitHub repository (Methods 4).

FIGS. 4A-4E illustrate evaluating performance of a CNN to predict the spatial coordinates of nuclei, mitochondria and cytoplasm according to Example 1. FIGS. 4A-4D: Representative paired sets from model training using nucleus (train (N=37)/test (N=5), mitochondria train (N=24)/test (N=5), and Cytoplasm train (N=61)/test (N=9) models showing bright-field input 402, target signal 404 and predicted signal 406 (FIG. 4A), with train/validation rolling mean square error loss graphs for nucleus (FIG. 4B) mitochondria (FIG. 4C), and Cytoplasm (FIG. 4D). FIG. 4E: Correlation coefficient of target fluorescence (T) vs. predicted fluorescence (P) images paired with correlation coefficient from brightfield signal(S) for the organelles that were used to train the models.

FIGS. 5A-5D illustrate label-free quantification of nucleus, cytoplasm and mitochondria at single cell resolution according to Example 1. FIG. 5A: Predicted nuclei, mitochondria and cytoplasm signals. FIG. 5B: Nuclei, mitochondria and cytoplasm of three representative cells from G1 or S phase. FIG. 5C: Nuclei, mitochondria and cytoplasm of three representative cells from G2/M phase. (B,C) Every data point is color-coded according to its organelle class. FIG. 5D: Cell features quantified from label free imaging correlate with cell cycle classes as defined by FUCCI. Color code indicates field of view (1-4).

FIGS. 6A-6F illustrate characterization of discrete and continuous cell cycle progression in NCI-N87 with label free imaging according to Example 1. FIG. 6A: Supervised approach to predict discrete cell cycle state from nuclei, mitochondria and cytoplasm features. FIG. 6B: Performance of trained classifier on test set. FIG. 6C: Unsupervised approach to predict continuous cell cycle progression from nuclei, mitochondria and cytoplasm features. FIG. 6D: Pseudotime derived from label free imaging features is differentially distributed across the four FUCCI-informed cell cycle classes. FIG. 6E: representative cell across cell cycle: FUCCI. FIG. 6F: representative cell across cell cycle: label free. In FIG. 6E, FIG. 6F, x-axis indicates pseudotime.

FIGS. 7A-7C illustrate sequencing imaging integration according to Example 1. FIG. 7A: Sequencing derived pseudotime of cell cycle progression. FIG. 7B: Co-clustering of sequenced and imaged cells based on pseudotime. Color code indicates cluster membership. FIG. 7C: Correlation between pathway activity and imaging-derived features.

FIG. 8 illustrates an approach for cell cycle classification and assigning sequenced to imaged cells according to Example 1.

FIG. 9 illustrates Fucci-derived pseudotime is differentially distributed across the four cell cycle phases according to Example 1.

FIG. 10 illustrates pseudotime derived from label-free imaging is differentially distributed across the four cell cycle phases according to Example 1.

FIG. 11 illustrates cell features derived from label free imaging correlate with FUCCI derived cell cycle progression according to Example 1. These features were prioritized based on their differential distribution across FUCCI-derived cell cycle classes (Anova test: p-value<0.01 across all four fields of view).

FIG. 12 illustrates sequencing and imaging derived features are correlated according to Example 1.

FIG. 13 is Table 1, which shows a correlation between imaging and sequencing derived feature pairs according to Example 1. Only the top 150 most significant correlations are displayed in Table 1. P-values are Bonferroni-corrected.

FIG. 14 is Table 2, which shows the definition of imaging features according to Example 1.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of +20%, +10%, +5%, or +1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

A convolutional neural network (CNN) is a type of deep neural network that has been applied, for example, to image analysis applications. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks.

A support vector machine (SVM) is a supervised learning model that uses statistical learning frameworks to predict the probability of a target. This disclosure contemplates that the SVMs can be implemented using a computing device (e.g., a processing unit and memory as described herein). SVMs can be used for classification and regression tasks. SVMs are trained with a data set (also referred to herein as a “dataset”) to maximize or minimize an objective function, for example a measure of the SVM's performance, during training. SVMs are known in the art and are therefore not described in further detail herein.

It should be understood that ANN, CNN, and SVM are provided only as example machine learning models. This disclosure contemplates that the machine learning model can be other supervised learning models, semi-supervised learning models, or unsupervised learning models. Machine learning models are known in the art and are therefore not described in further detail herein.

### Example Methods

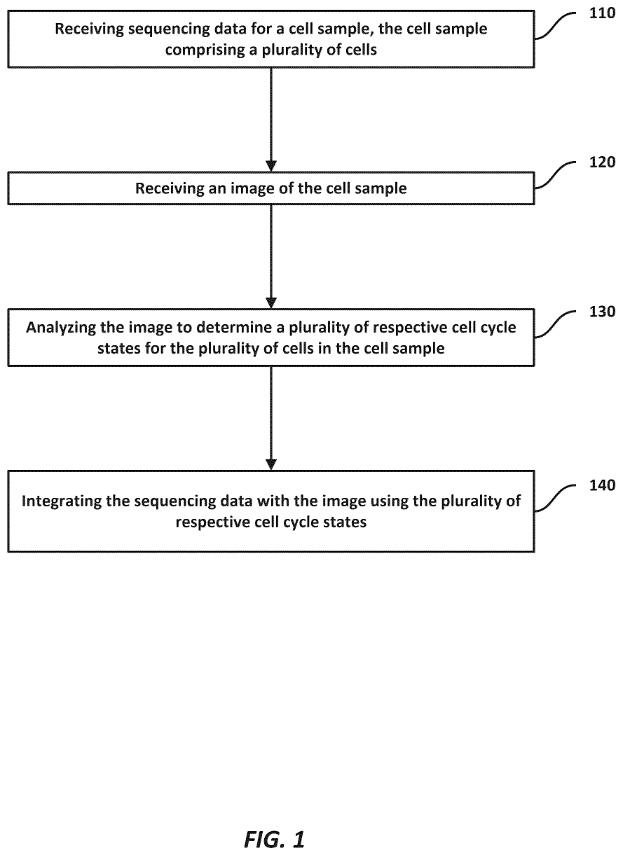

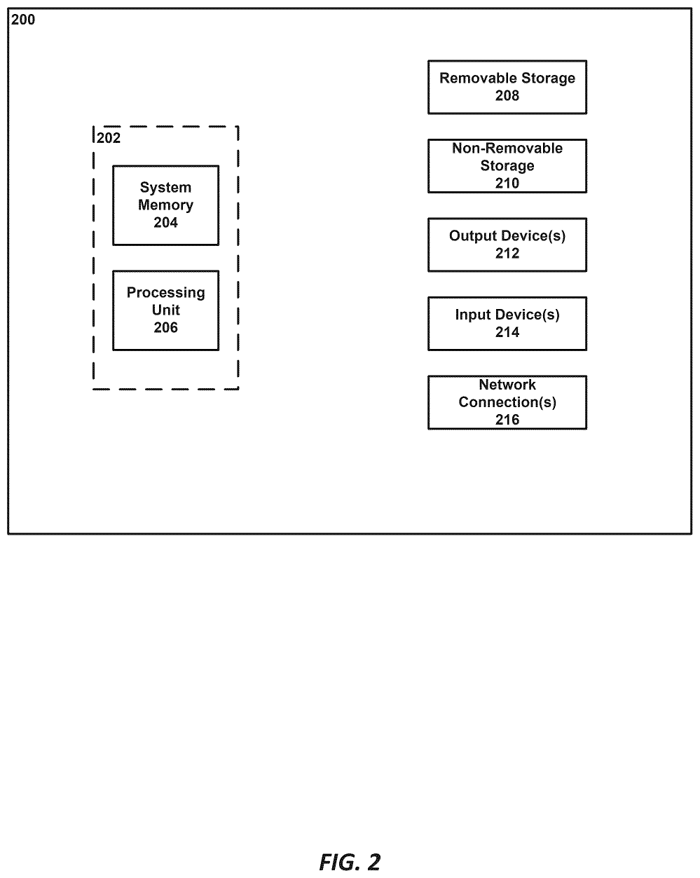

FIG. 1 is a flowchart of an example method for integrating imaging and sequencing data using cell cycle states according to implementations described herein. This disclosure contemplates that the operations of FIG. 1 can be performed using at least a processor and memory, for example at least a processor and memory as described with regard to the computing device of FIG. 2.

At step 110, the method includes receiving sequencing data for a cell sample. The cell sample includes a plurality of cells. Optionally, in some implementations, the cell sample is a cell cluster. As used herein, sequencing data refers to raw data generated by sequencing technologies, such as DNA sequencing (e.g., whole-genome sequencing, exome sequencing) and RNA sequencing. It includes the nucleotide sequences of the entire genome or specific regions of interest. Sequencing data is used to study genetic variations, mutations, and other features of the genome. Additionally, the sequencing data is single-cell sequencing data refers to the genomic information obtained from individual cells rather than from bulk tissue samples that contain many cell. In other words, single-cell sequencing data for one or more individual cells in the cell sample is received at step 110. In the Examples, the cells are cancer cells, specifically cells from stomach cancer cell line (NCI-N87). In addition, the sequencing data is single-cell RNA sequencing (scRNA-seq) of NCI-N87 cells. It should be understood that stomach cancer cells are provided only as an example. This disclosure contemplates that the cells may be other types of cells, including but not limited to, stomach cancer cells.

At step 120, the method includes receiving an image of the cell sample. As described above, the cell sample optionally includes cancer cells, specifically NCI-N87 cells. Optionally, in some implementations, the image is a fluorescently-labeled image. As used herein, a fluorescently-labeled image is a type of microscopic image in which specific structures, molecules, or cells are tagged with fluorescent dyes or proteins. When exposed to light of a specific wavelength, these fluorescent labels emit light at a different wavelength, allowing the labeled structures to be visualized with high contrast against a dark background. In the Examples, the fluorescently-labeled image is an image generated using the Fluorescence Ubiquitination Cell Cycle Indicator (FUCCI) system. This system is used to visualize cell cycle progression in living cells through fluorescent markers. It should be understood that FUCCI images are provided only as an example. This disclosure contemplates that the fluorescently-labeled image may be another type of labeled image. Optionally, in other implementations, the image is a brightfield image. As used herein, a brightfield image is a type of microscopic image produced using brightfield microscopy, where light passes directly through a specimen. The image is formed by the contrast between the specimen and the surrounding light In the Examples, the image is a 3D brightfield image.

At step 130, the method includes analyzing the image to determine a plurality of respective cell cycle states for the plurality of cells in the cell sample. Optionally, the plurality of respective cell cycle states include one or more of G1 Phase, S Phase, G2 Phase, M Phase, and G0 Phase. As used herein, cell cycle states define the stages that a cell goes through to grow and divide. Such stages include G1 Phase (Gap 1), where the cell grows and carries out normal functions and/or prepares for DNA replication; S Phase (Synthesis), where DNA replication occurs, resulting in the duplication of the cell's genetic material; G2 Phase (Gap 2), where the cell continues to grow and prepares for mitosis, ensuring all DNA is replicated and undamaged; M Phase (Mitosis), where the cell divides its copied DNA and cytoplasm to form two daughter cells; and G0 Phase, where a resting or quiescent stage where cells exit the cycle and do not actively divide.

As described above, the image can be a brightfield image in some implementations. In these implementations, the step of analyzing the image to determine the plurality of respective cell cycle states for the plurality of cells in the cell sample includes using a trained machine learning model. The trained machine learning model can be a supervised machine learning model. Optionally, the trained machine learning model is a CNN. In the Examples, the trained machine learning model is a CNN, specifically U-Net.

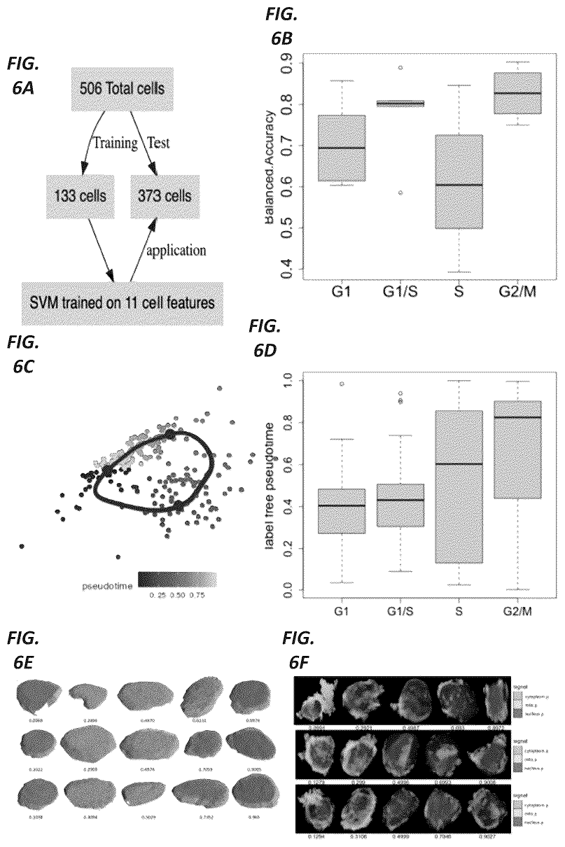

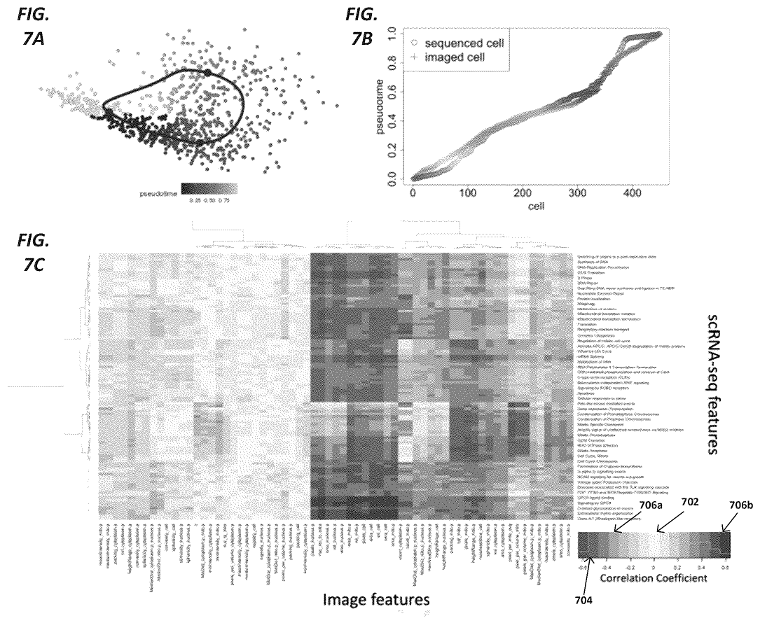

As described above, machine learning models such as CNNs are trained with a dataset to maximize or minimize an objective function. For example, a CNN can be trained using a training dataset including brightfield images (see e.g. images 402 in FIG. 4A) and corresponding fluorescent images (see e.g. 404 in FIG. 4A). The corresponding fluorescent images serve as labels during training. Thus, the CNN can be trained to predict spatial distributions of organelles of cells in simulated images (see e.g. 406 in FIG. 4A) using the training dataset. Accordingly, the CNN, when deployed in inference mode, is configured to infer a spatial distributions of organelles of a cell in a simulated image in response to processing an input brightfield image. In other words, in inference mode, the trained CNN extracts features from the input brightfield image and outputs the spatial distributions of organelles of the cell in the simulated image (i.e. the target).

In some implementations, the spatial distributions of organelles of a cell, which are predicted by the trained machine learning model, are the coordinates of organelles of the cell. As used herein, organelles can include, but are not limited to, nuclei, mitochondria, cytoplasm, cell walls, cell membranes, or any other organelle. Optionally, the organelles include nucleus and mitochondria. Optionally, the organelles include nucleus, mitochondria, and cytoplasm. For example, the trained machine learning model (i.e. U-Net) in the Examples below predicts the coordinates of organelles such as the nucleus and mitochondria.

The method can further include segmenting one or more organelles of the plurality of cells in the simulated image of the cell sample. It should be understood that one or more organelles can be segmented from each of the plurality of cells in the cell sample. This disclosure contemplates using a known segmentation technique to segment organelles. In the Examples below, the organelles such as nuclei and mitochondria predicted by the trained CNN are segmented using Cellpose, which is a generalist CNN model based on U-Net architecture with residual blocks that segments cells from a wide range of image types including 2D and 3D images. It should be understood that Cellpose is provided only as an example segmentation technique. This disclosure contemplates using segmentation techniques other than Cellpose.

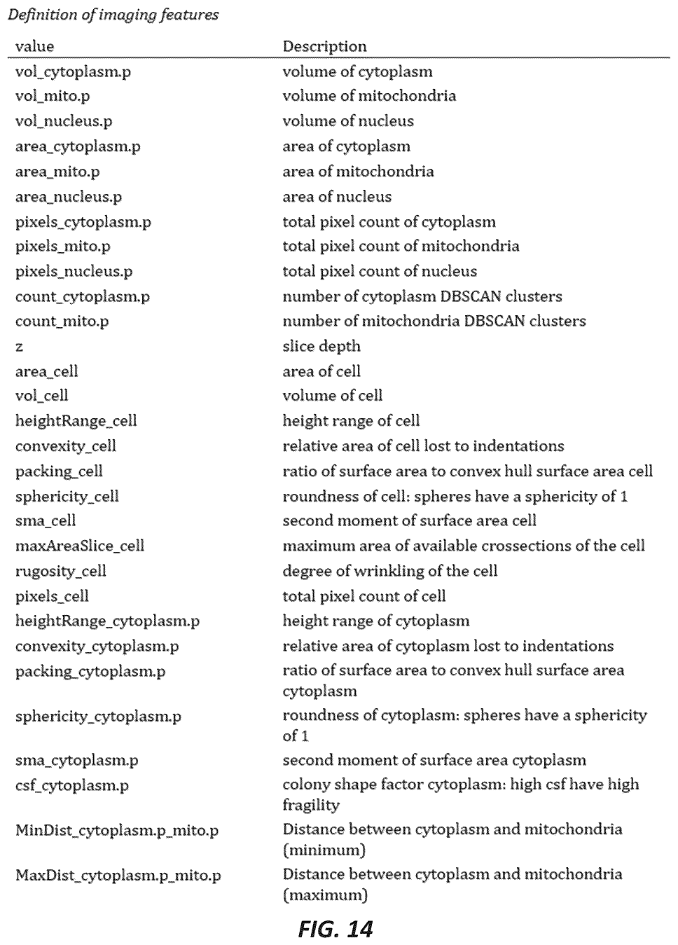

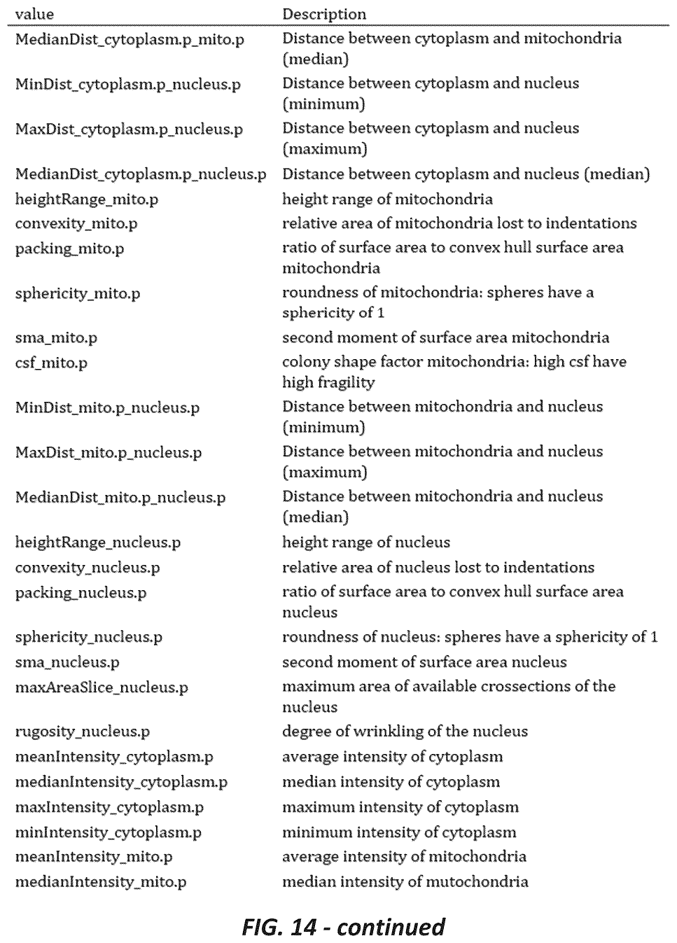

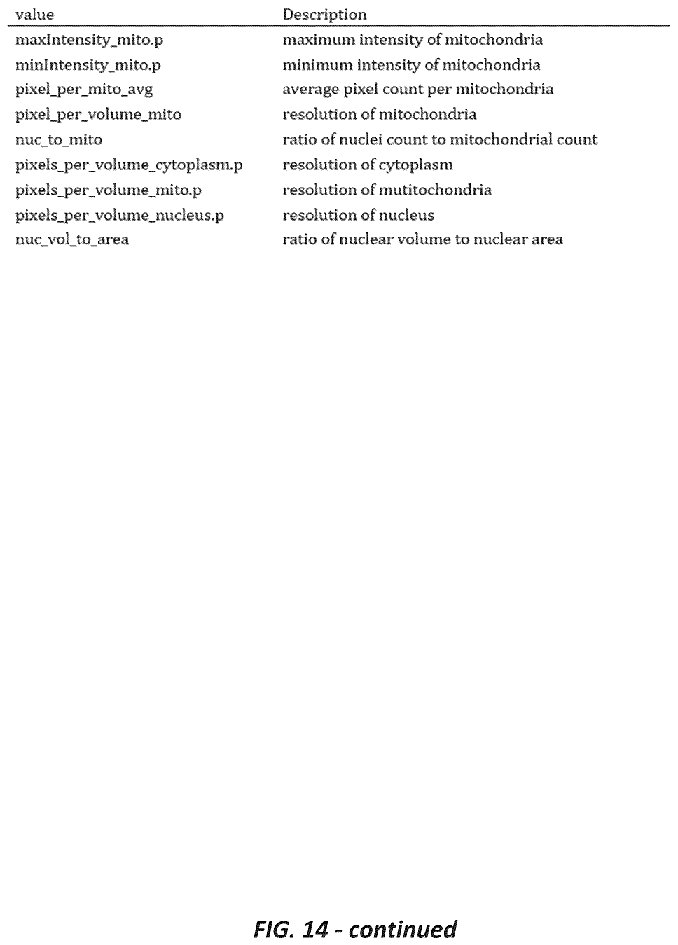

The method can further include quantifying a plurality of cell features of the plurality of cells in the simulated image of the cell sample. It should be understood that a plurality of features can be quantified from each of the plurality of cells in the cell sample. Example cell features shown in FIG. 14, which includes more than fifty features, can be quantified. In some implementations, a subset of the features shown in FIG. 14 can be quantified. For example, the quantified cell features can include the following eleven features (which are demonstrated as correlating with cell cycle state in the Examples): area of cell, area of nucleus, number of cytoplasm density-based clustering algorithm (DBSCAN) clusters, number of mitochondria DBSCAN clusters, maximum area of available cross sections of the nucleus, ratio of nuclear volume to nuclear area, total pixel count of cell, total pixel count of mitochondria, total pixel count of nucleus, volume of cell, and volume of nucleus.

The method can further include correlating the plurality of cell features with a cell cycle state. It should be understood that the plurality of cell features for each of the plurality of cells in the cell sample can be correlated with a respective cell cycle state. Correlating the plurality of cell features with the cell cycle state can include prioritizing features for classification of cell cycle state. For example, the correlation can be accomplished by inferring a cell cycle pseudotime for a cell using one or more of the plurality of cell features, where the plurality of cell features are correlated with the cell cycle state using the cell cycle pseudotime.

At step 140, the method includes integrating the sequencing data with the image using the plurality of respective cell cycle states. In some implementations, this step includes mapping each of the plurality of cells in the image to a set of the plurality of cells in the sequencing data using the plurality of respective cell cycle states. In other implementations, this step includes mapping each of the plurality of cells in the sequencing data to a set of the plurality of cells in the image using the plurality of respective cell cycle states. In yet other implementations, this step includes: mapping each of the plurality of cells in the image to a set of the plurality of cells in the sequencing data using the plurality of respective cell cycle states; and mapping each of the plurality of cells in the sequencing data to a set of the plurality of cells in the image using the plurality of respective cell cycle states.

In some aspects, the techniques described herein relate to a method including: integrating sequencing data for a cell sample with an image as described above with regard to FIG. 1; and providing a diagnosis, prognosis, or treatment recommendation for a subject based on the integrated sequencing data and image of the cell sample.

In some aspects, the techniques described herein relate to a method including: integrating sequencing data for a cell sample with an image as described above with regard to FIG. 1; and administering a treatment to a subject based on the integrated sequencing data and image of the cell sample.

### Example Computing Device

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by box 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

A cell's transcriptome is a channel of information propagation; it is a snapshot of how a cell interacts with and responds to its environment. Cells co-existing in the same tumor, or in the same cell line, often differ in their genomes and transcriptomes. We and others have shown that these differences often correlate with morphological and structural differences between cells and several imaging- and transcriptome-derived feature pairs have been identified. Expression of cell membrane- and cell surface genes can inform the size of a cell. The copy number of mitochondrial DNA (mtDNA) can serve as proxy for inter-cell differences in the number of mitochondria they carry and several regulators of cell shape and morphology have been identified, including: FLO11, STE2, ELN, and TGFB1.

New microfluidic platforms have been designed to link phenotypic analysis of living cells to single-cell sequencing. However, the throughput of these platforms is limited to a few hundred cells, precluding learning from these data, general rules that link fitness to transcriptome snapshots. SCOPE-seq-a microwell array based platform has been developed and claims that a more aggressive cell loading of the platform could increase throughput to several thousands of cells. Nevertheless, all solutions available so far that link phenotypic and genomic measurement, have done so over a narrow time window: typically less than one cell generation.

Proposed herein is in-silico mapping of sequenced and imaged cells as a solution to extend the temporal reach of transcriptome-phenotype integration. We leverage the influence cell cycle progression has on a cell's transcriptome, morphology and subcellular organization to integrate transcriptome profiles obtained from scRNA-seq of a stomach cancer cell line (NCI-N87) with 3D brightfield images from the same cell line. This example focuses on a prerequisite for integrating sequencing and imaging data: inferring a cell's position along the cell cycle continuum from 3D images. Our example is structured as follows: we first evaluate whether the Fluorescent Ubiquitination-based Cell Cycle Indicator (FUCCI) can inform cell cycle progression at a higher temporal resolution than simply distinguishing G1 and S/G2/M phases of the cell cycle. We then use a convolutional neural network (CNN), to calculate the spatial coordinates of nucleus, mitochondria and cytoplasm in each imaged cell. Next we use size and shape statistics calculated for these subcellular compartments to assign cells to a continuum of cell cycle progression. We conclude with an outlook on how the assigned cell cycle state can be used to link imaged cells to sequenced cells.

Projecting a snapshot of the transcriptome onto subcellular architecture is a natural way to integrate information on the pathway membership of genes and their localization into a holistic view of a cell.

### Results

Continuous Temporal Resolution on Cell Cycle Progression with FUCCI

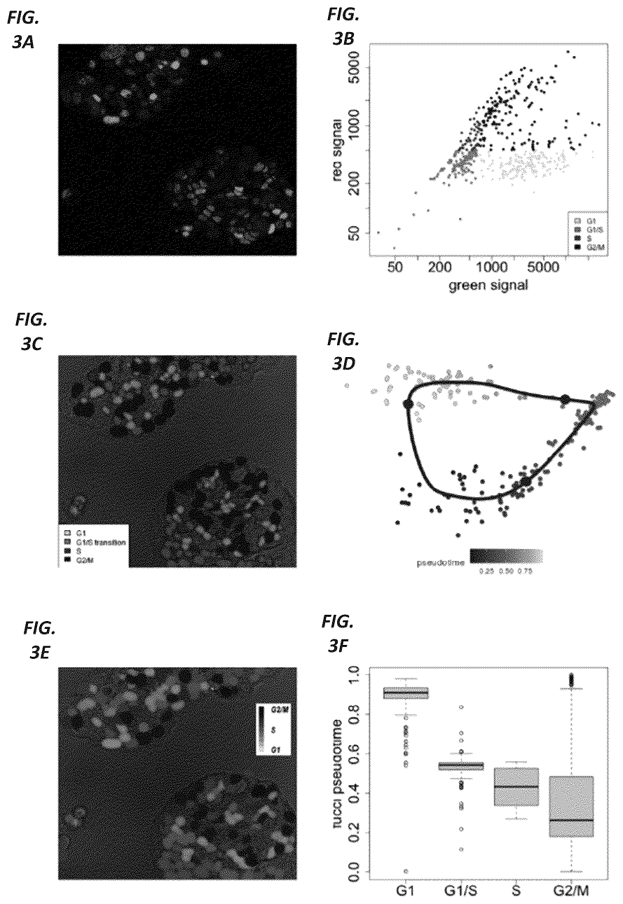

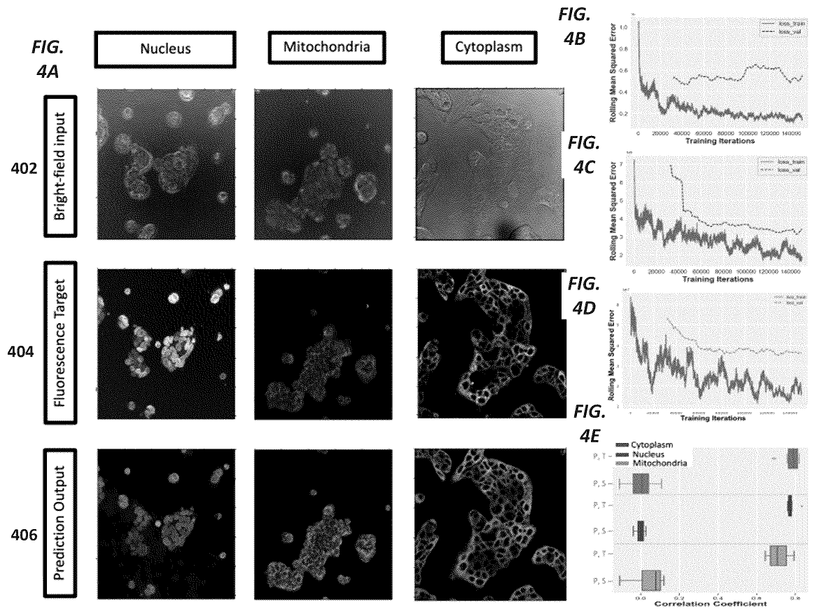

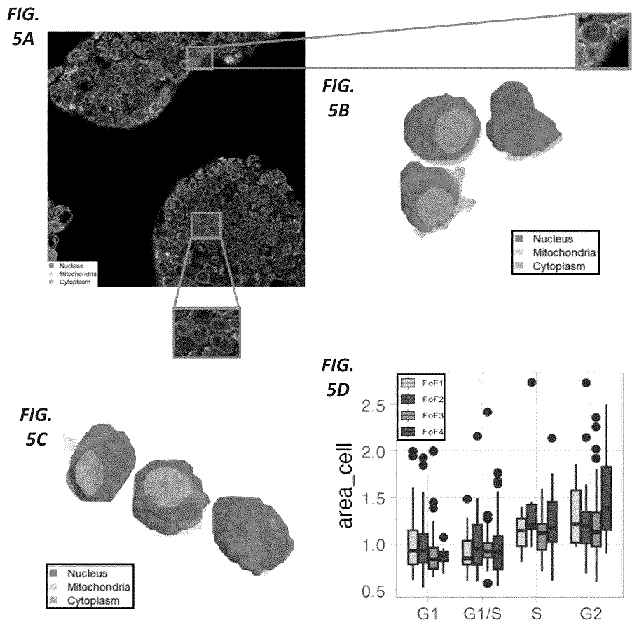

We established NCI-N87 cells stably transfected with FUCCI-vector plasmids (see Methods). We acquired 3D images of the transfected cells on a Leica TCS SP8 equipped with an oil-immersion objective (see Methods) on brightfield and green/red fluorescence channels (FIG. 3A). Green cells were classified as cells in G1 phase and comprised 30% of the population. Red cells and double positive (yellow) cells were classified as cells in the S phase (9.5% cells) and G2/M phase, respectively (32% of the population). Cells with no color were assigned as G1/S transitional and comprised 28% of the population (FIG. 3B, 3C).

Each cell cycle phase has been described as a series of steps that proceeds at a fixed rate. Biologically, the steps refer to a sequence of events that need to be completed for the cell to proceed to the next cell cycle phase (e.g. accumulation of a molecular factor or degradation of proteins). We asked whether FUCCI could quantify cell cycle progression at a temporal resolution higher than that given by distinction of the four cell cycle phases. Specifically, we asked whether FUCCI reporters combined with 3D imaging can rank cells according to how many of the steps within a given cell cycle phase each cell has completed. We calculated 45 intensity features for each cell across the three imaging channels and projected them onto PCA space as input to the Angle method for inferring pseudotime trajectories (FIG. 3D). This method computes the angle with respect to the origin in a two-dimensional PCA space and uses this angle as pseudotime for generating a cyclical trajectory. The inferred pseudotime was correlated with and differentially distributed across the four discrete cell cycle classes (FIGS. 3E, 3F).

These results indicate that unsupervised methods can be applied to FUCCI based features to widen the application of FUCCI to continuous cell cycle mapping.

### Mitochondrial, Cytoplasmic and Nuclear Changes Inform Cell Cycle Progression

Despite having revolutionized live-cell imaging of cell cycle transitions, imaging FUCCI labeled cells requires excitation light, which can cause photobleaching and phototoxicity. We therefore asked if a similar continuous cell cycle mapping could be achieved with label-free imaging.

We used 3D images 402, 404 of FIG. 4A containing the nuclei, mitochondria or cytoplasm of NCI-N87 cells (see Methods, cell culture), to train a previously developed label-free U-Net convolutional neural network (CNN). U-Net is an encoder-decoder artificial neural network for semantic segmentation. Models were trained to predict the coordinates of nucleus, mitochondria and cytoplasm respectively (see Methods) shown in images 406 of FIG. 4A. Model performance was evaluated using the Pearson correlation coefficient between the pixel intensities of the model's predicted output and the independent fluorescence images, with all three signals scoring well above background (Pearson r>0.71 for signal vs. Pearson r<0.04 for background; Wilcoxon rank sum test P<0.002; FIGS. 4A-4E). The segmentation of nuclei was done using Cellpose—a general purpose algorithm for cell segmentation (see Methods), while mitochondrial and cytoplasmic signals were assigned to nuclei with DBSCAN (see Methods).

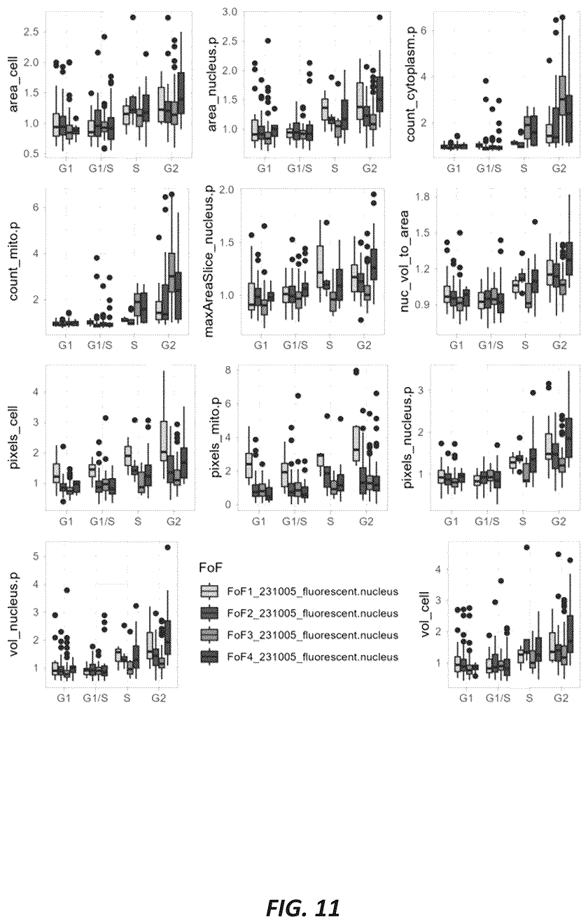

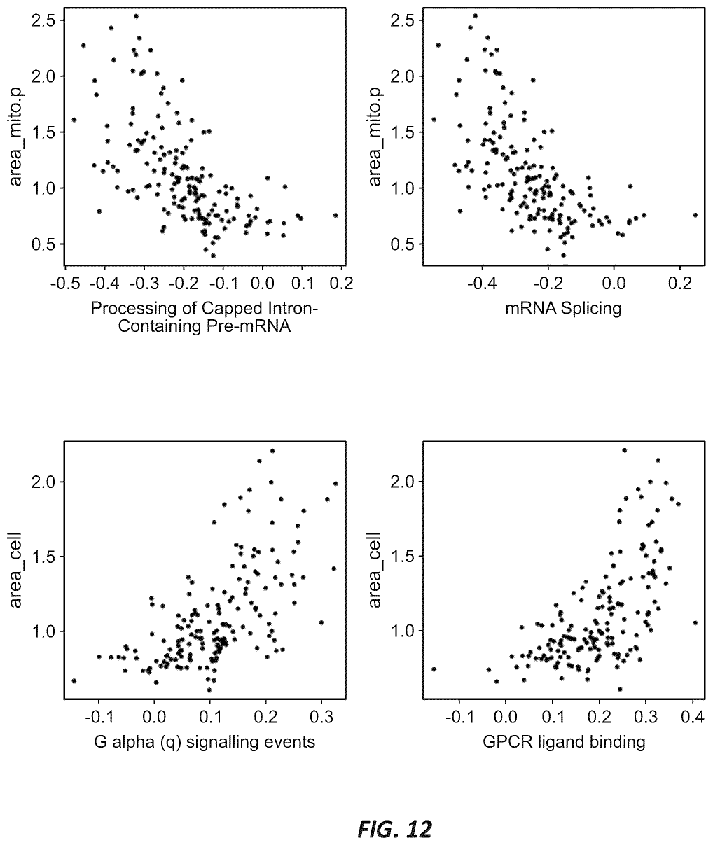

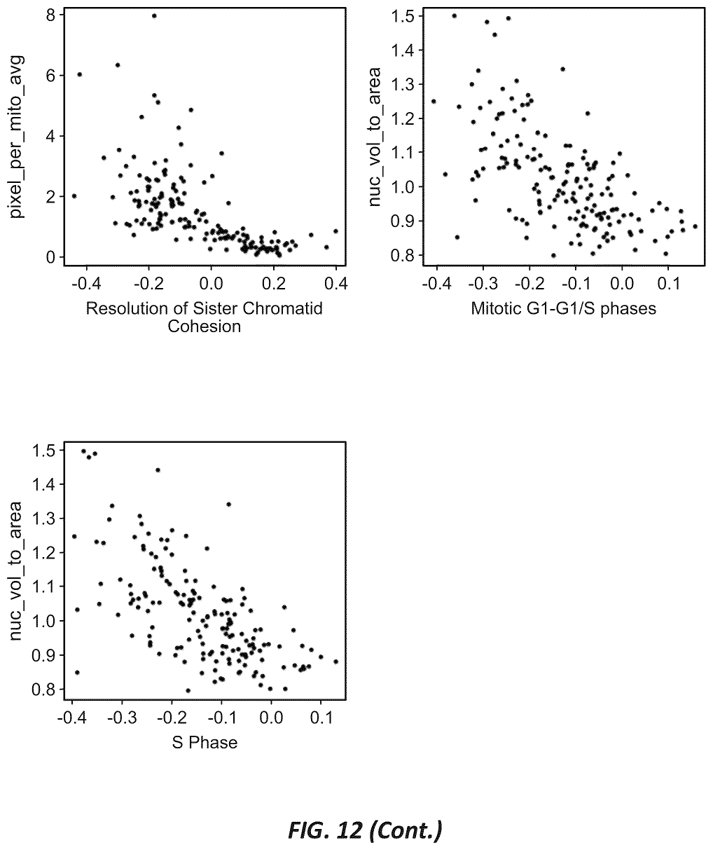

The trained model was used to predict the coordinates of these structures in NCI-N87 cells (FIGS. 5A-5C), allowing quantification of 65 organelle features (FIG. 14), including their volumes, areas and symmetry. We identified 11 of these features which differed significantly across cell cycle classes as defined by FUCCI across all images (Anova p-value≤0.01; FIG. 5D, FIG. 11, FIG. 14), suggesting that changes in these structures during the cell cycle are quantifiable with label-free imaging. These 11 features are: area of cell, area of nucleus, number of cytoplasm density-based clustering algorithm (DBSCAN) clusters, number of mitochondria DBSCAN clusters, maximum area of available cross sections of the nucleus, ratio of nuclear volume to nuclear area, total pixel count of cell, total pixel count of mitochondria, total pixel count of nucleus, volume of cell, and volume of nucleus.

As a first test of this hypothesis, we used the FUCCI-based classification to train an SVM to predict discrete cell cycle state from the 11 nuclei-, mitochondria- and cytoplasm features. The performance of the trained classifier was evaluated on an independent test set consisting of 373 unseen cells (FIG. 6A). The classifier achieved an average accuracy >0.7 across the four images for G1, G1/S transitional and G2M cells (FIG. 6B). Classification accuracy was lower (median≈0.6) for S-phase (FIG. 6B).

We also used the 11 cell organelle features as input to the Angle method for inferring pseudotime trajectories (FIG. 6C). Similar to the FUCCI-derived pseudotime, the pseudotime inferred from label-free imaging was also correlated with and differentially distributed across the four discrete cell cycle classes (FIG. 6D).

Compared to FUCCI-derived pseudotime, pseudotime inferred from label-free imaging had a higher variance, possibly indicating a higher temporal resolution on the cell cycle. This approach allowed us to view virtual animations of the cell cycle by sampling cells representative across the entire pseudotime spectrum (FIGS. 6E, 6F).

### Cell Cycle Pseudotime Links Imaged to Sequenced Cells

Knowing a cell's precise point on the cell cycle continuum is a novel opportunity to accomplish a challenging goal: integrating live-cell imaging with single-cell sequencing.

Well established and widely adopted methods for pseudotime inference from sequencing data exist and have been described in detail elsewhere, e.g. These provide the opportunity to use the pseudotime derived herein from imaging in order to map imaged cells onto sequenced cells. To achieve this, we used single cell RNA sequencing (scRNAseq) data previously published for NCI-N87. A total of 1,076 genes involved in cell cycle, with highly variable expression among the 738 sequenced NCI-N87 cells, were prioritized for pseudotime inference with the Angle method (FIG. 7A).

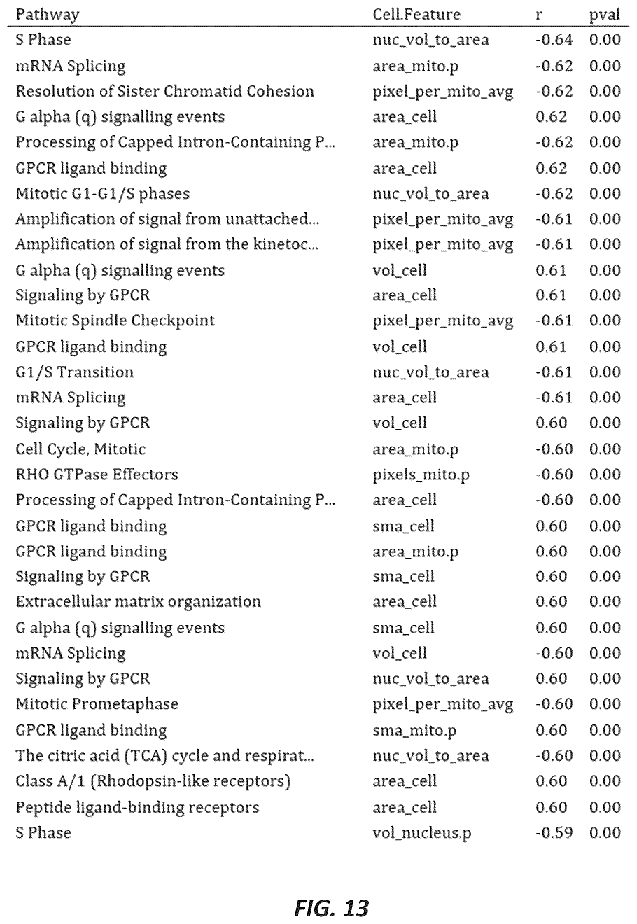

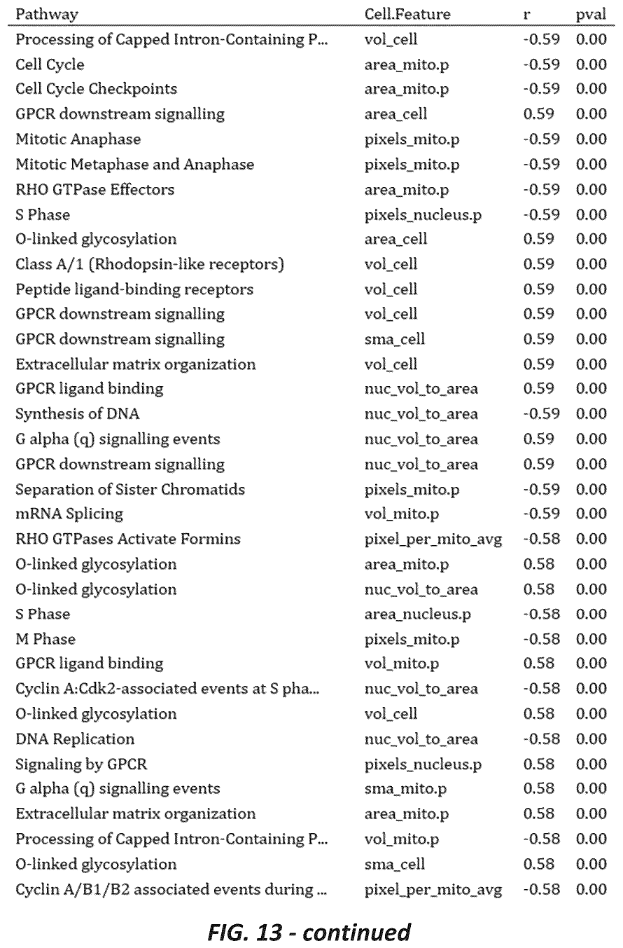

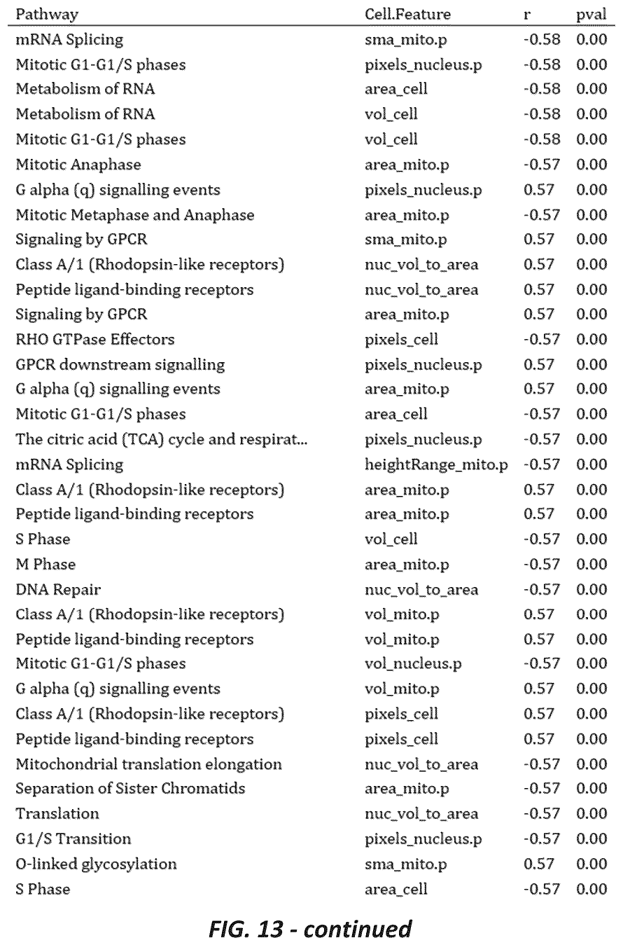

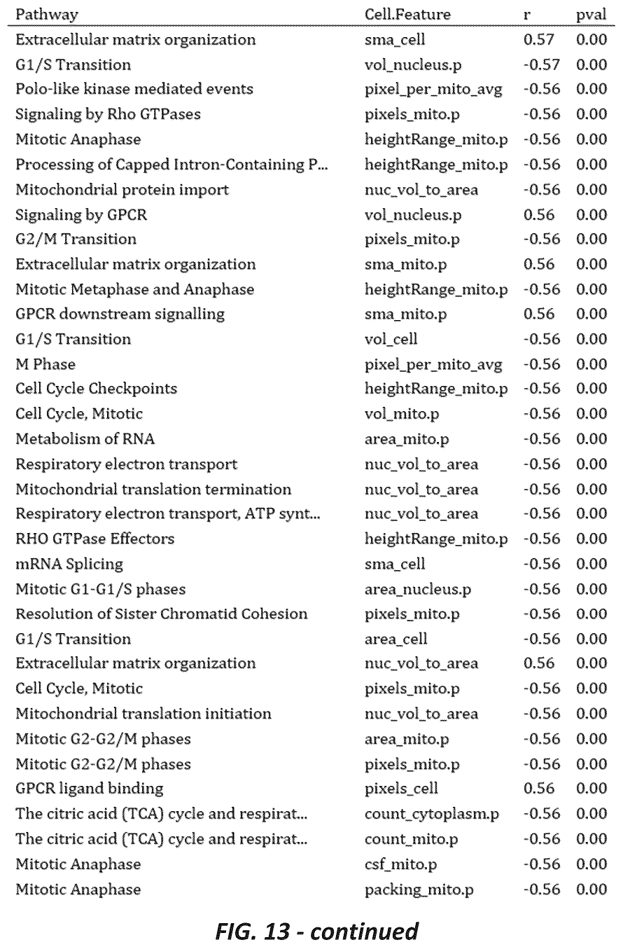

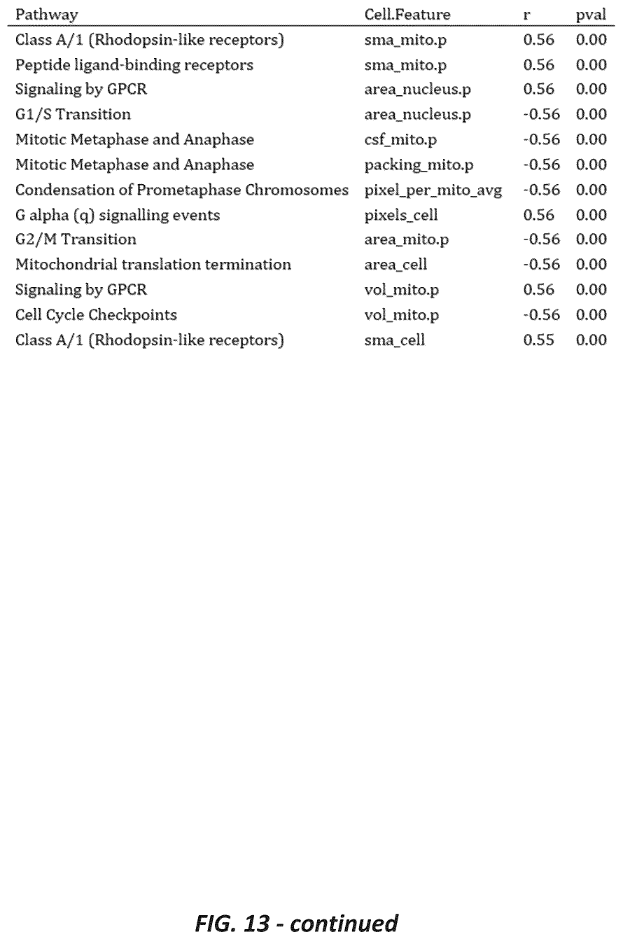

The distributions of sequencing- and imaging-derived pseudotimes were similar, and were co-clustered using DBSCAN (FIG. 7B and Methods). This approach identified 225 clusters, 99% of which contained both, sequenced and imaged cell representatives (FIG. 7B), further referred to as multi-type clusters. The average number of sequenced and imaged cells per cluster was 3.4 and 2.4 respectively. For each multi-type cluster we calculated the mean pathway activity among sequenced cell members and the mean organelle features among imaged cell members. Comparing all imaging- and sequencing derived feature pairs across clusters identified 4,484 significantly correlated feature pairs (Pearson r≥0.38; Bonferroni-corrected p-value<0.05; FIG. 7C and FIG. 13).

Of the top 150 pathways with the highest correlation coefficients, 24 were Signaling by GPCR, GPCR ligand binding, or GPCR downstream signaling; 18 were involved in G1, S or G1/S transition; and 18 were stages of mitosis. GPCR pathways had positive correlations with the area and volume of the cell, mitochondria and nucleus, as well as the nuclear volume to area ratio. G1/S pathways had negative correlations with the area and volume of the cell, mitochondria and the nuclear volume to area ratio. M pathways had a strong negative correlation with the area of mitochondria.

Overall, associations between pathway activities and cell morphology were evident in three large clusters. Pathways from the first cluster (indicated in yellow (see heatmap scale 702) in FIG. 7C), are largely responsible for the formation of the extracellular matrix. These pathways also contribute to the organization of the ECM and collagen production. There are elements of cell growth and proliferation regulation, in part by downregulating the P13K/AKT pathway. Cells which express these pathways tend to be large and round and have large volume and area of the nucleus, mitochondria, cytoplasm.

The second cluster included pathways that were almost entirely dedicated to mitosis (indicated in blue (see heatmap scale 704) in FIG. 7C). They regulate the G2/M transition, each stage of M phase, the actin cytoskeleton components; the packaging, cohesion, and separation of sister chromatids; as well as other aspects of the mitotic spindle and chromosome organization. Cells which have high expression of these pathways will have slightly higher cytoplasmic and mitochondrial intensities, lower mitochondrial resolution, and elevated mitochondrial counts. Indeed, the number of mitochondria has been described to increase during G2 and M phases compared to G1/S due to the fusion of mitochondria in G1/S and subsequent fission in G2/M.

The third cluster (indicated in red and green (see heatmap scale 706a and 706b) in FIG. 7C), consisted of pathways that play a major role in cell cycle transitioning and replication of genetic material. A selection of these elements includes the conversion from S to M by Orc1 removal and DNA synthesis, replication, damage recognition, and repair; mRNA processing, apoptosis, environmental responsiveness, angiogenesis and motility, and cell cycle protein regulation. Cells expressing these pathways were smaller, had a low maximum intensity of mitochondria, low area of cytoplasm, low convexity of mitochondria, and substantially lower volume and area of the mitochondria and nucleus.

Taken together these associations between imaging- and transcriptome derived features pairs are in line with decades of research unraveling how the transcriptome influences cell shape, scaling, compartmentalization and protein localization.

### Discussion

A combination of unsupervised and supervised classification methods have been developed to classify imaged cells to one of three possible cell cycle states (G1, S or G2M). Some studies however suggest that gene expression signatures of cell state transitions occur as a continuous process, rather than in abrupt steps. We have shown that both, 3D imaging of FUCCI cells as well as a high resolution on the 3D subcellular architecture of cells can provide a quantitative description of cell cycle progression, allowing us to move beyond the classification of cells into discrete states. While it is unclear which of the two—FUCCI- or organelle features—provide a higher temporal resolution on cell cycle progression, it is noteworthy that quantification of FUCCI features per nucleus were dependent on the accurate segmentation of nuclei, which in turn were derived from semantic segmentation of label-free images. Ultimately, it is reasonable to hypothesize that the highest temporal resolution can be achieved by combining both methodologies. Verifying this hypothesis will require applying the approach presented herein to a live cell imaging experiment spanning multiple days.

In contrast to imaging data, for which methods for classification of cell cycle state are scarce, several methods for cell cycle inference from sequencing data exist and are widely adopted. We have for the first time integrated sequencing and imaging derived cell cycle pseudotimes for mapping clusters of imaged cells to sequenced cell clusters from the same gastric cancer cell line. For this experiment, sequenced and imaged cells were obtained from different timepoints. The next step will be to repeat this approach within a longer-term live cell imaging experiments, wherein cells are sampled intermittently for sequencing. Knowing the entire transcriptome of a cell will always require killing the cell. The ability to assign the transcriptome state of a lysed cell to its closest living relative (which is still actively growing and expanding), would be unprecedented and would open the door for genotype-phenotype mapping at single cell resolution, forward in time. The growing field of spatial transcriptomics, while simplifying mapping between imaged cells and their transcriptomes, cannot accomplish this particular task because it requires killing all spatially adjacent cells for sequencing. This means that spatial transcriptomics cannot be used for learning to predict phenotypes forward in time, but could only be leveraged for retrospective phenotypic interpretation. The phenotypic interpretation of genomes and transcriptomes is the bottleneck to progress in medicine, it lags far behind manipulation and quantification. By understanding how the transcriptomes and genomes of co-existing cells diverge, how these divergent populations compete or cooperate, one can learn to predict the long-term consequence of exposing them to different therapeutic environments.

### Methods

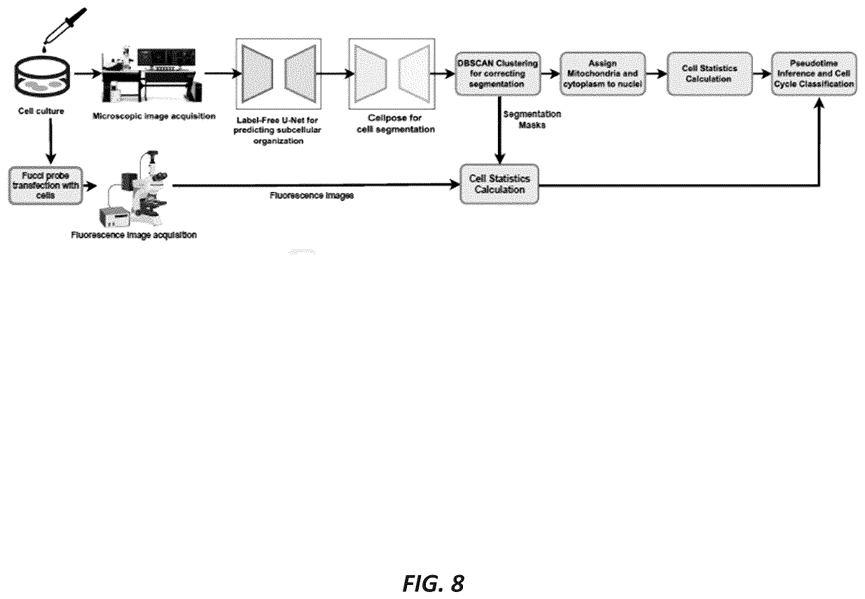

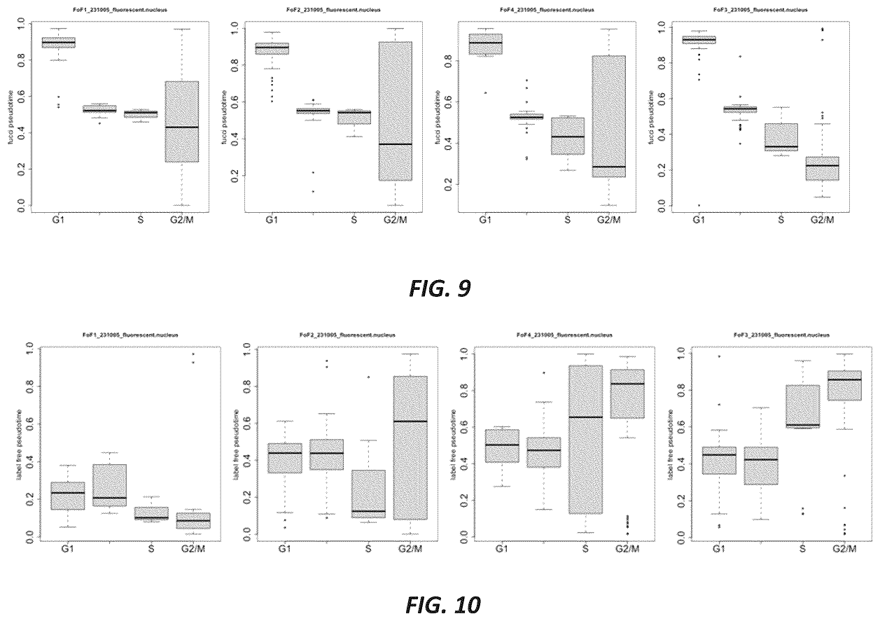

Our proposed approach for inferring cell's position along the cell cycle continuum from 3D images is depicted in FIG. 8. In the following subsections, we illustrate each component of the proposed approach.

Transfection of NCI-N87 Cells with FUCCI-Vector Plasmids

For cell cycle-phase visualization, lentiviral FUCCI (fluorescent ubiquitination-based cell cycle indicator) expression system was used. The PIP Fucci vector (Addgene, Plasmid #118616) encoding the FUCCI probe was co-transfected with the packaging plasmids into HEK 293T cells. Supernatant from the culture medium containing high-titer viral solutions were collected and used for transduction into NCI-N87 cells. PIP Fucci labels G1 cells with mVenus (green) and S-G2-M cells with (mCherry). Cells with stable integration of the plasmid were established by FACS with both green and red channels.

### Cell Culture

NCI-N87 were seeded in μ-Slide 8 Well, ibiTreat-Tissue Culture Treated Polymer Coverslip (Fisher Scientific) using 5×10⁴ or 1×10⁵ cells in 300 ul of RPMI-1640 with 10% FBS and 1% penicillin streptomycin. Cells were treated with 0.3 ul or 0.6 ul BioTracker 488 Nuclear per 300 ul for 3 hrs or 70-120 nM BioTracker 405 Mito for 6 hrs (Millipore Sigma). Cells were washed 3× with warm PBS and resuspended in complete growth media for imaging.

### Microscopy

A confocal microscope (Leica TCS SP8) equipped with a 63×/1.4-NA oil-immersion objective (Leica Apochromat×100/1.4 W) was used for image acquisition. The 3D cell images were recorded in LAS×3.5.7 using Photomultiplier Tube detectors, resulting in a pixel size of 0.232 μm and Z-interval of 0.29 μm. We collected 70 z slices of target fluorescence dye (cytoplasm, mitochondria or nuclei) and brightfield signal using a 400 Hz scan speed. For each field of view, we imaged an area of 56,644 μm², which took approximately 3 minutes for the brightfield and the fluorescence channels.

### Training and Application of a U-Net Convolutional Neural Network to Predict Subcellular Organization

Stacks of 70 brightfield and fluorescence 16-bit images were processed into “.ome.tif” files containing NumPy arrays. We trained a previously developed label-free U-Net convolutional neural network, on these 3D images containing the nuclei or mitochondria of NCI-N87 to calculate the spatial coordinates of nucleus and mitochondria in each imaged cell. All models were trained using number of batches=8 for 3D patches of 128×128×32p³ (XYZ), the Adam optimizer with a learning rate of 0.001 and with beta 1 and 2 of 0.9 and 0.999 respectively for 150,000 minibatch iterations. The model training pipeline was implemented in Python using PyTorch on a Nvidia DGX A100 Tesla V100.

The trained model was applied to 3D brightfield live-cell imaging of NCI-N87 cells for nine hours at a three hour interval, acquiring a total of four images. Image acquisition was performed as described above. The nuclei and mitochondria predicted by the trained model were segmented using Cellpose—a generalist model that segments cells from a wide range of image types including 2D and 3D images. Cellpose is based on a U-Net architecture with residual blocks. In 3D cell segmentation, testing of the fine-tuned pretrained Cellpose model (i.e., cytotorch_2) was completed where a gradient was generated for xy, yz, xz slices, independently. Then the gradient was averaged to obtain the final prediction. This approach allows testing 3D images on a 2D based deep learning model.

### Correcting Nuclei Segmentation

We correct segmentation by merging IDs belonging to the same cell using 3D imaging data. We read the center coordinates of cells from a CSV file and scale the z-coordinates according to the specified Z-stack distance. Using DBSCAN clustering on the x, y, and z coordinates, we identify clusters of points representing individual cells, filtering out noise.

For each identified cell cluster, the function gathers coordinates associated with the new cell from the original segmentation files. If a cell does not have sufficient z-stack representation, it is excluded. The function then calculates the centroids of the newly identified cells.

### Assigning Mitochondria and Cytoplasm to Nuclei

We assign mitochondrial and cytoplasmic compartments to nuclei based on 3D imaging data. For each organelle (mitochondria, cytoplasm), the function reads the corresponding TIF image, identifies pixels with intensity above the 90th percentile, and records their coordinates and signal values. Nucleus coordinate files are then loaded and associated with their respective cells.

For each nucleus, we expand the bounding box around it, identify organelle pixels within this expanded region, and assign these pixels to the nucleus. DBSCAN clustering is performed on the combined coordinates (nucleus, mitochondria, and cytoplasm) for each cell. The cluster containing the nucleus is identified and organelle pixels in this cluster are assigned to the corresponding cell. We correct for doubly assigned coordinates by removing ambiguous assignments. This method ensures accurate spatial assignment of mitochondrial and cytoplasmic compartments to individual nuclei for further analysis.

### Cell Statistics Calculation

Statistics are calculated for each cell based on its segmented coordinates and signal intensities. Statistics related to nucleus shape and size are computed, including area, volume, fractal dimension, rugosity, and height range. Next, organelle-specific statistics are computed. For each signal type (e.g., mitochondria, cytoplasm), we iterate through unique organelles within the cell and computes statistics related to shape, size, and spatial relationships, including area, volume, convexity, packing, sphericity, and distance to other organelles, as well as intensity-based features (mean, median, maximum, and minimum intensity values). R packages ‘geometry’, ‘habtools’ and ‘misc3d’ are used to calculate these statistics.

Additional statistics calculated include average pixels per mitochondria, pixel density per volume for each organelle, and ratios between organelle volumes.

### Pseudotime Inference

Cell features derived from either i) FUCCI or from ii) label-free imaging are used on multiple fields of view to infer pseudotime. For both i) and ii), each feature vector is divided by its median across all cells and log-transformed prior to inference. Trajectory inference is conducted using the Angle method, while employing principal component analysis (PCA) for dimensionality reduction. To evaluate potential batch effects, pseudotime inference is performed on all fields of view combined as well as on each field of view separately.

### Training an SVM to Classify Cells to Cell Cycle Phases

We perform cell cycle phase classification using Support Vector Machine (SVM) and evaluate the model's performance as follows. An SVM with a radial basis kernel is trained using cell features derived from label free imaging as input, and the four cell cycle classes (inferred from FUCCI imaging) as labels. This trained SVM model is subsequently used to predict cell cycle phases on test data from different cells across all available fields of view.

Quantification of Pathway Activity from Gene Expression

Gene set variation analysis (GSVA) is performed to compute the activities of 1,119 pathways from the REACTOME database, based on single-cell RNA sequencing (scRNA-seq) of 738 NCI-N87 cells. R-function “gsva” was used from the R-package GSVA.

### Assigning Sequenced to Imaged Cells Based on Pseudotime

This segment aims to co-cluster image and sequencing statistics for further analysis. Pseudotimes inferred from sequencing (P-seq) and from imaging (P-img) are combined into a one-dimensional vector and a density-based clustering algorithm (DBSCAN) is applied to the combined vector (R-function ‘dbscan::dbscan’ is used with eps=0.001 and minPts=2). Clusters that consist solely of one data type (either “P-seq” or “P-img”) and noise are filtered out.

For each remaining cluster we calculate two averages: the average pathway activity profile across all sequenced cell members of that cluster and the average organelle feature profile across all imaged cell members of that cluster. Subsequently, Pearson correlation coefficients between all possible feature-pairs are calculated.

### Example 2

Mapping sequenced to imaged cells (Example 1) enables training an ANN to predict cell phenotypes (such as cell migration-, proliferation- and death rates, which typically require live-cell imaging at multiple timepoints for computation) from sequencing data acquired at a single timepoint. We will further refer to an ANN trained to predict phenotypes from sequencing data as pheno-ANN. Pheno-ANNs would have broad applicability in the clinical setting. For example, gastric cancers most commonly metastasize to the peritoneum, liver, bone, lymph nodes and lung. Trastuzumab, a monoclonal antibody targeting the HER2 receptor, is the first targeted therapy shown to improve the prognosis of metastatic gastric cancer patients without increasing side effects. Patients with amplified HER2 benefit from anti-HER2 therapy. However, retrospective analysis of clinical data from a cohort of breast cancer patients controversially suggested that HER2-negative cancers would also benefit from Trastuzumab. A follow-up study confirmed the finding, additionally revealing that metastatic site context may account for Trastuzumab efficacy in HER2-negative breast cancer. Whether this applies in gastric cancer as well is unknown.

We propose an experiment designed to mimic this clinical scenario, wherein neo-adjuvant and adjuvant therapy both include Trastuzumab. We focus on 6 metastatic stomach cancer cell lines we recently characterized. The metastatic site of origin, HER2-amplification status, and sensitivity to anti-HER2 therapy differs between these cell lines. Sequencing the DNA and RNA of thousands of representatives from these cell lines, we classified cells into groups with unique karyotype profiles. We will grow colonies from representatives of these subpopulations. Hereby each growth environment will be optimized to activate receptors that are over-expressed in the corresponding metastatic tissue site compared to the primary tumor site (i.e. stomach). We will further refer to a group of cells descending from the same ancestral single cell as clone. We will monitor the cell cycle progression profiles of each clone for up to 11 generations. We will use a snapshot of transcriptome changes taken at generation six, to predict cell cycle progression in subsequent generations. We will then evaluate the potential of environmental changes in cell densities and subpopulation frequencies, to extend the temporal reach of predictions.

We will expose ten HER2-positive PDXs to a minimum of four doses of Trastuzumab. A control group will receive a non-tumor binding isotype. Doses will be administered once a week, will range from 3 mg/kg to 15 mg/kg and dosing will begin when tumor volumes reach approximately 200 mm³. Tumors will be biopsied four days after first exposure to Trastuzumab. We will sequence the transcriptomes of 20,000 single cells derived from these biopsies. Cells will be clustered according to their transcriptomes and the RNA profile of each cluster will be used as input to our model to predict cell cycle behavior following Trastuzumab exposure. Comparing the predicted tumor growth rate to that observed will inform the accuracy of phenotypic predictions.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method comprising:
receiving sequencing data for a cell sample, the cell sample comprising a plurality of cells;
receiving an image of the cell sample;
analyzing the image to determine a plurality of respective cell cycle states for the plurality of cells in the cell sample; and
integrating the sequencing data with the image using the plurality of respective cell cycle states.

2. The computer-implemented method of claim 1, wherein the step of integrating the sequencing data with the image comprises: mapping each of the plurality of cells in the image to a set of the plurality of cells in the sequencing data using the plurality of respective cell cycle states; and mapping each of the plurality of cells in the sequencing data to a set of the plurality of cells in the image using the plurality of respective cell cycle states.

3. The computer-implemented method of claim 1, wherein the image is a brightfield image.

4. The computer-implemented method of claim 3, wherein analyzing the image to determine the plurality of respective cell cycle states for the plurality of cells in the cell sample comprises using a trained machine learning model.

5. The computer-implemented method of claim 4, wherein using the trained machine learning model comprises:
inputting the brightfield image into the trained machine learning model; and
outputting a spatial distribution of organelles of the plurality of cells in a simulated image of the cell sample from the trained machine learning model.

6. The computer-implemented method of claim 5, further comprising segmenting one or more organelles of the plurality of cells in the simulated image of the cell sample.

7. The computer-implemented method of claim 6, further comprising quantifying a plurality of cell features of the plurality of cells in the simulated image of the cell sample.

8. The computer-implemented method of claim 7, wherein the plurality of cell features comprise area of cell, area of nucleus, number of cytoplasm density-based clustering algorithm (DBSCAN) clusters, number of mitochondria DBSCAN clusters, maximum area of available cross sections of the nucleus, ratio of nuclear volume to nuclear area, total pixel count of cell, total pixel count of mitochondria, total pixel count of nucleus, volume of cell, and volume of nucleus.

9. The computer-implemented method of claim 8, further comprising correlating the plurality of cell features with a cell cycle state.

10. The computer-implemented method of claim 9, wherein correlating the plurality of cell features with the cell cycle state comprises inferring a cell cycle pseudotime for a cell using one or more of the plurality of cell features, wherein the plurality of cell features are correlated with the cell cycle state using the cell cycle pseudotime.

11. The computer-implemented method of claim 4, further comprising:
providing a training dataset comprising brightfield images and corresponding fluorescent images; and
training a machine learning model to predict spatial distributions of organelles of cells in simulated images using the training dataset.

12. The computer-implemented method of claim 1, wherein the image is a fluorescently-labeled image.

13. The computer-implemented method of claim 1, wherein the plurality of respective cell cycle states comprise one or more of G1 Phase, S Phase, G2 Phase, M Phase, and G0 Phase.

14. A method comprising:
integrating sequencing data for a cell sample with an image of the cell sample according to the computer-implemented method of claim 1; and
providing a diagnosis, prognosis, or treatment recommendation for a subject based on the integrated sequencing data and image of the cell sample.

15. A method comprising:
integrating sequencing data for a cell sample with an image of the cell sample according to the computer-implemented method of claim 1; and
administering a treatment to a subject based on the integrated sequencing data and image of the cell sample.

16. A computer system comprising:
one or more processors and one or more computer-readable memories operably coupled to the one or more processors, the one or more computer-readable memories having instructions stored thereon that, when executed by the one or more processors, cause the computer system to perform a method comprising:
receiving sequencing data for a cell sample, the cell sample comprising a plurality of cells;
receiving an image of the cell sample;
analyzing the image to determine a plurality of respective cell cycle states for the plurality of cells in the cell sample; and
integrating the sequencing data with the image using the plurality of respective cell cycle states.

17. The computer system of claim 16, wherein the image is a brightfield image.

18. The computer system of claim 17, wherein analyzing the image to determine the plurality of respective cell cycle states for the plurality of cells in the cell sample comprises using a trained machine learning model.

19. The computer system of claim 18, wherein using the trained machine learning model comprises:
inputting the brightfield image into the trained machine learning model; and
outputting a spatial distribution of organelles of the plurality of cells in a simulated image of the cell sample from the trained machine learning model.

20. The computer system of claim 19, further comprising quantifying a plurality of cell features of the plurality of cells in the simulated image of the cell sample, wherein the plurality of cell features comprise area of cell, area of nucleus, number of cytoplasm density-based clustering algorithm (DBSCAN) clusters, number of mitochondria DBSCAN clusters, maximum area of available cross sections of the nucleus, ratio of nuclear volume to nuclear area, total pixel count of cell, total pixel count of mitochondria, total pixel count of nucleus, volume of cell, and volume of nucleus.
